(19) United States
(12) Patent Application Publication
(10) Pub. No.: US 2024/0043364 A1
(43) Pub. Date: Feb. 8, 2024

(54) METHOD FOR PREPARING 3,3',4,4'-DICYCLOHEXYLTETRACARBOXYLIC ACID AND METHOD FOR TREATING ACIDIC WASTEWATER

(71) Applicant: HEBEI HAILI FRAGRANCES CO., LTD, Hebei (CN)

(72) Inventors: Shuai SHAO, Hebei (CN); Yuntang ZHANG,, Hebei (CN); Wenge LI, Hebei (CN); Yufen ZHANG, Hebei (CN); Jingxiao WANG, Hebei (CN); Feiyang WANG, Hebei (CN); Qin YAN, Hebei (CN); Xiao WANG, Hebei (CN); Tiecheng LIU, Hebei (CN)

(21) Appl. No.: 17/641,092

(22) PCT Filed: Aug. 23, 2021
(86) PCT No.: PCT/CN2021/113953

§ 371 Date: Mar. 7, 2022

(51) Int. Cl. C07C 51/09; B01J 23/46; C02F 1/52; C07C 67/303

(52) U.S. Cl. CPC C07C 51/09; B01J 23/462; B01J 23/464; C02F 1/5245; C07C 67/303; C07C 2602/28; C02F 2103/36

(57) ABSTRACT

A method for preparing 3,3′,4,4′-dicyclohexyltetracarboxylic acid and a method for treating an acidic wastewater are provided. In the preparation method according to the present disclosure, a ruthenium-rhodium mixed catalyst is used to catalyze the hydrogenation reduction reaction, which improves the selectivity of the reaction and reduces the generation of isomers, thereby reducing the generation of by-products and increasing the purity of 3,3′,4,4′-dicyclohexyltetracarboxylic acid. The results of the examples show that the purity of 3,3′,4,4′-dicyclohexyltetracarboxylic acid prepared by the method according to the present disclosure is not less than 99.62%.

### CROSS-REFERENCE TO RELATED APPLICATIONS

The present application is a National Stage of PCT International Application Serial Number PCT/CN2021/113953, filed Aug. 23, 2021, the disclosures of which are incorporated by reference herein.

The present disclosure relates to the technical field of organic synthesis, and in particular to a method for preparing 3,3′,4,4′-dicyclohexyltetracarboxylic acid and a method for treating an acidic wastewater.

Polyimides obtained by 1,2,4,5-cyclohexyltetracarboxylic dianhydride have a higher glass transition temperature and initial pyrolysis temperature than those obtained by the aliphatic tetracarboxylic dianhydride, but they are very brittle. Polyimides prepared by 3,3′,4,4′-dicyclohexyltetracarboxylic acid have advantages of excellent heat resistance, mechanical properties, flexibility, and solubility. Therefore, there is a great demand for 3,3′,4,4′-dicyclohexyltetracarboxylic acid.

At present, 3,3′,4,4′-dicyclohexyltetracarboxylic acid is prepared by hydrogenation reduction of 3,3′,4,4′-biphenyltetracarboxylic di-anhydride in the presence of palladium-carbon catalyst, but there are many by-products, including 3,3′,4,4′-diphenyl phthalic acid, 3,3′,4,4′-diphenyl phthalate, etc., which severely affect the purity of 3,3′,4,4′-dicyclohexyltetracarboxylic acid, and make the purity of 3,3′,4,4′-dicyclohexyltetracarboxylic acid less than 98.5%, thereby severely restricting the industrialization of this product.

### SUMMARY OF THE INVENTION

In view of this, the purpose of the present disclosure is to provide a method for preparing 3,3′,4,4′-dicyclohexyltetracarboxylic acid and a method for treating an acidic wastewater. 3,3′,4,4′-dicyclohexyltetracarboxylic acid prepared by the preparation method provided by the present disclosure has high purity.

In order to achieve the above purpose, the present disclosure provides the following technical solutions:

The present disclosure provides a method for preparing 3,3′,4,4′-dicyclohexyltetracarboxylic acid, comprising the following steps:
mixing 3,3′,4,4′-biphenyltetracarboxylic acid and an alcohol, and subjecting the resulting mixture to an esterification reaction to obtain an esterification reaction system;
mixing the esterification reaction system and a ruthenium-rhodium mixed catalyst, and subjecting the resulting mixture to a hydrogenation reaction in a hydrogen atmosphere to obtain a hydrogenation reaction system; and
subjecting the hydrogenation reaction system to a hydrolysis reaction to obtain 3,3′,4,4′-dicyclohexyltetracarboxylic acid,
wherein the ruthenium-rhodium mixed catalyst is a mixed catalyst of a ruthenium-containing catalyst and a rhodium-containing catalyst, and the mixed catalyst has a mass ratio of the ruthenium-containing catalyst to the rhodium-containing catalyst of 1:6;
the ruthenium-containing catalyst is a ruthenium-carbon catalyst, and the ruthenium-carbon catalyst has a mass percentage of ruthenium of 5%;
the rhodium-containing catalyst is a rhodium-carbon catalyst, and the rhodium-carbon catalyst has a mass percentage of rhodium of 3%.

In some embodiments, the 3,3′,4,4′-biphenyltetracarboxylic acid has a purity of not less than 99.8%.

In some embodiments, the alcohol comprises a monohydric alcohol and/or a dihydric alcohol, and the alcohol has a water content of not more than 2%.

In some embodiments, the monohydric alcohol comprises one or more selected from the group consisting of methanol, ethanol and isopropanol.

In some embodiments, the dihydric alcohol comprises 1,3-propanediol.

In some embodiments, the esterification reaction is conducted at a temperature of ° C. and a pH value of 0.5-1.0 for 3-6 h.

In some embodiments, a mass ratio of the 3,3′,4,4′-biphenyltetracarboxylic acid to the ruthenium-rhodium mixed catalyst is in a range of 1:(0.02-0.05).

In some embodiments, the hydrogenation reaction is conducted at a temperature of 80-90° C. and a pressure of 0.2-0.5 MPa.

In some embodiments, the hydrolysis reaction comprises:
subjecting a hydrogenation reaction material liquid obtained after the hydrogenation reaction to a solid-liquid separation to obtain a solid catalyst and a hydrogenation reaction system;
adjusting the hydrogenation reaction system to a pH value of 9-11, and subjecting the hydrogenation reaction system to a first hydrolysis to obtain a first hydrolysis system;
adjusting the first hydrolysis system to a pH value of 1-2, and subjecting the first hydrolysis system to a second hydrolysis.

In some embodiments, the first hydrolysis is conducted at a temperature of ° C. for 1-3 h.

In some embodiments, the hydrolysis reaction further comprises subjecting a second hydrolyzed material liquid obtained after the second hydrolysis to a solid-liquid separation to obtain a first acidic wastewater and a crude product; and
washing and drying the crude product to obtain a pure product of 3,3′,4,4′-dicyclohexyltetracarboxylic acid,
wherein a reagent for washing comprises water;
the washing also results in a second acidic wastewater.

The present disclosure also provides a method for treating an acidic wastewater, comprising the following steps:
mixing an acidic wastewater with an aluminum-iron mixed powder, and subjecting the resulting mixture to a complexation reaction to obtain a complexation reaction system; and
mixing the complexation reaction system with a polyvinyl alcohol, and subjecting the resulting mixture to a precipitation reaction to obtain a purified acidic water,
wherein the acidic wastewater comprises the first acidic wastewater and/or the second acidic wastewater obtained by the method in the above technical solutions.

In some embodiments, the aluminum-iron mixed powder has a mass ratio of iron to aluminum of 1:(1-3).

In some embodiments, a mass ratio of the acidic wastewater to the aluminum-iron mixed powder is in a range of 1:(0.001-0.02).

In some embodiments, the complexation reaction is conducted for 0.5-2 h.

In some embodiments, a mass ratio of the acidic wastewater to the polyvinyl alcohol is in a range of 1:(0.001-0.02).

In some embodiments, the precipitation reaction is conducted at a pH value of 6-7 for 0.5-2 h.

In some embodiments, the method further comprises subjecting a precipitation reaction material liquid obtained after the precipitation reaction to a solid-liquid separation to obtain the purified acidic wastewater; and
distilling the purified acidic wastewater to obtain a distillate and a solid of salts.

The present disclosure provides a method for preparing 3,3′,4,4′-dicyclohexyltetracarboxylic acid, comprising the following steps: mixing 3,3′,4,4′-biphenyltetracarboxylic acid and an alcohol, and subjecting the resulting mixture to an esterification reaction to obtain an esterification reaction system; mixing the esterification reaction system and a ruthenium-rhodium mixed catalyst, and subjecting the resulting mixture to a hydrogenation reaction in a hydrogen atmosphere to obtain a hydrogenation reaction system; and subjecting the hydrogenation reaction system to a hydrolysis reaction to obtain 3,3′,4,4′-dicyclohexyltetracarboxylic acid, wherein the ruthenium-rhodium mixed catalyst is a mixed catalyst of a ruthenium-containing catalyst and a rhodium-containing catalyst, and the mixed catalyst has a mass ratio of the ruthenium-containing catalyst to the rhodium-containing catalyst of 1:6, the ruthenium-containing catalyst comprises a ruthenium-carbon catalyst, and the ruthenium-carbon catalyst has a mass percentage of ruthenium of 5%, the rhodium-containing catalyst comprises a rhodium-carbon catalyst, and the rhodium-carbon catalyst has a mass percentage of rhodium of 3%. In the preparation method according to the present disclosure, a ruthenium-rhodium mixed catalyst is used to catalyze the hydrogenation reduction reaction, which improves the selectivity of the reaction and reduces the generation of isomers, thereby reducing the generation of by-products and increasing the purity of 3,3′,4,4′-dicyclohexyltetracarboxylic acid. The results of the examples show that the purity of 3,3′,4,4′-dicyclohexyltetracarboxylic acid prepared by the method according to the present disclosure is not less than 99.62%.

Meanwhile, the use of the above mixed catalyst of ruthenium-containing catalyst and rhodium-containing catalyst makes the conditions of the hydrogenation reaction mild, and the hydrogenation reaction could be carried out at a temperature of 80-90° C. and a pressure of 0.2-0.5 MPa.

Furthermore, in the present disclosure, 3,3′,4,4′-dicyclohexyltetracarboxylic acid with a purity of not less than 99.8% is used as a raw material, which also reduces the generation of isomers and further improves the purity of the final 3,3′,4,4′-dicyclohexyltetracarboxylic acid, so that the purity of 3,3′,4,4′-dicyclohexyltetracarboxylic acid is improved to a range of 99.94-99.96%.

The present disclosure also provides a method for treating an acidic wastewater obtained by the preparation method described in the above technical solutions. The treatment method provided by the present disclosure could recover sodium chloride from the acidic wastewater, the content of TOC in the sodium chloride is low, and moreover the method is simple in operation, and could significantly reduce the COD value of the acidic wastewater, reaching the class IV of Environmental Quality Standard for Surface Water, thereby greatly improving the energy saving and environmental protection of the preparation method.

### BRIEF DESCRIPTION OF THE DRAWINGS

FIG. 1 is the nuclear magnetic resonance (NMR) spectrum of 3,3′,4,4′-dicyclohexyltetracarboxylic acid obtained in Example 1.

FIG. 2 is the NMR spectrum of 3,3′,4,4′-dicyclohexyltetracarboxylic acid obtained in Example 2.

FIG. 3 is the NMR spectrum of 3,3′,4,4′-dicyclohexyltetracarboxylic acid obtained in Example 3.

FIG. 4 is the NMR spectrum of 3,3′,4,4′-dicyclohexyltetracarboxylic acid obtained in Comparative Example 1.

### DETAILED DESCRIPTION OF THE EMBODIMENTS

The present disclosure provides a method for preparing 3,3′,4,4′-dicyclohexyltetracarboxylic acid, comprising the following steps:
mixing 3,3′,4,4′-biphenyltetracarboxylic acid and an alcohol, and subjecting the resulting mixture to an esterification reaction to obtain an esterification reaction system;
mixing the esterification reaction system and a ruthenium-rhodium mixed catalyst, and subjecting the resulting mixture to a hydrogenation reaction in a hydrogen atmosphere to obtain a hydrogenation reaction system; and
subjecting the hydrogenation reaction system to a hydrolysis reaction to obtain 3,3′,4,4′-dicyclohexyltetracarboxylic acid.

In the present disclosure, unless otherwise specified, the raw materials used are preferably commercially available products.

In the present disclosure, 3,3′,4,4′-biphenyltetracarboxylic acid and an alcohol are mixed, and the resulting mixture is subjected to an esterification reaction to obtain an esterification reaction system.

In some embodiments of the present disclosure, 3,3′,4,4′-biphenyltetracarboxylic acid has a purity of not less than 98.5%, preferably 98.5-99.8%, and more preferably 99.8%. In some embodiments of the present disclosure, 3,3′,4,4′-biphenyltetracarboxylic acid is purchased from Hebei Haili flavor Co., Ltd., China. In the present disclosure, 3,3′,4,4′-biphenyltetracarboxylic acid with a purity of not less than 98.5% is used to increase the purity of 3,3′,4,4′-dicyclohexyltetracarboxylic acid; further, under the condition that the purity of 3,3′,4,4′-biphenyltetracarboxylic acid is 99.8%, the purity of the obtained 3,3′,4,4′-dicyclohexyltetracarboxylic acid is further improved.

In some embodiments of the present disclosure, the alcohol comprises a monohydric alcohol and/or a dihydric alcohol, wherein the monohydric alcohol comprises one or more selected from the group consisting of methanol, ethanol and isopropanol, and the dihydric alcohol comprises 1,3-propanediol; the alcohol has a water content of not more than 2%.

In some embodiments of the present disclosure, a ratio of the molar amount of the carboxyl group in 3,3′,4,4′-biphenyltetracarboxylic acid to the molar amount of the hydroxyl group in the alcohol is 1:1.

In some embodiments of the present disclosure, the esterification reaction is conducted at a temperature of 80-100° C., preferably 85-95° C., and more preferably 90° C.; the esterification reaction is conducted for 3-6 h, preferably 4-5 h. In some embodiments of the present disclosure, the esterification reaction is conducted at a pH value of 0.5-1.0. In some embodiments of the present disclosure, the pH value of the esterification reaction is adjusted by an acid solution, and the acid solution comprises hydrochloric acid. In the present disclosure, there is no limitation on the concentration and dosage of the hydrochloric acid, as long as the pH value could be adjusted to 1-2.

In the present disclosure, after the esterification reaction system is obtained, the esterification reaction system and the ruthenium-rhodium mixed catalyst are mixed and a hydrogenation reaction is conducted in a hydrogen atmosphere to obtain a hydrogenation reaction system.

In some embodiments of the present disclosure, the ruthenium-rhodium mixed catalyst is a mixed catalyst of a ruthenium-containing catalyst and a rhodium-containing catalyst. In some embodiments, the mixed catalyst has a mass ratio of the ruthenium-containing catalyst to the rhodium-containing catalyst of 1:6. In some embodiments, the ruthenium-containing catalyst is a ruthenium-carbon catalyst, and the ruthenium-carbon catalyst has a mass percentage of ruthenium of 5%. In some embodiments, the rhodium-containing catalyst is a rhodium-carbon catalyst, and the rhodium-carbon catalyst has a mass percentage of rhodium of 3%. In the specific embodiments of the present disclosure, the ruthenium-carbon catalyst and the rhodium-carbon catalyst are purchased from Kaili New Materials Co., Ltd, Xi'an, China.

In some embodiments of the present disclosure, a mass ratio of 3,3′,4,4′-biphenyltetracarboxylic acid to ruthenium-rhodium mixed catalyst is in a range of 1:(0.02-0.05).

In some embodiments of the present disclosure, the hydrogenation reaction is conducted at a temperature of 80-90° C., preferably 85° C.; the hydrogenation reaction is conducted at a pressure of 0.2-0.5 MPa, and preferably 0.3-0.4 MPa. In the present disclosure, there is no specific limitation on the time of the hydrogenation reaction, as long as the hydrogenation reaction system no longer absorbs hydrogen.

In some embodiments of the present disclosure, the method further comprises subjecting a hydrogenation reaction material liquid obtained after the hydrogenation reaction to a solid-liquid separation to obtain a solid catalyst and a hydrogenation reaction system. In some embodiments of the present disclosure, the solid-liquid separation is conducted by filtration.

In the present disclosure, after obtaining the hydrogenation reaction system, the hydrogenation reaction system is subjected to a hydrolysis reaction to obtain 3,3′,4,4′-dicyclohexyltetracarboxylic acid.

In some embodiments of the present disclosure, the hydrolysis reaction comprises:
adjusting the hydrogenation reaction system to a pH value of 9-11, and subjecting the hydrogenation reaction system to a first hydrolysis to obtain a first hydrolysis system; and
adjusting the first hydrolysis system to a pH value of 1-2, and subjecting the first hydrolysis system to a second hydrolysis.

In some embodiments of the present disclosure, the hydrogenation reaction system is adjusted to a pH value of 9-11, and subjected to the first hydrolysis to obtain the first hydrolysis system.

In the present disclosure, a reagent for adjusting the hydrogenation reaction system to a pH value of 9-11 comprises caustic soda flakes.

In some embodiments of the present disclosure, the first hydrolysis is conducted at a temperature of 80-90° C. for 1-3 h, and preferably 2 h.

In the present disclosure, the method further comprises: cooling and filtering a first hydrolyzed material liquid obtained after the first hydrolysis to obtain a first hydrolysis system and an alkaline wastewater. In some embodiments of the present disclosure, the alkaline wastewater is refluxed to the esterification reaction stage for use.

In the present disclosure, after obtaining the first hydrolysis system, the first hydrolysis system is adjusted to a pH value of 1-2, and subjected to a second hydrolysis.

In some embodiments of the present disclosure, the second hydrolysis comprises the following steps: dissolving the first hydrolysis system in water, and then adjusting to a pH value of 1-2. In the present disclosure, there is no specific limitation on the ratio of water to the first hydrolysis system, as long as the first hydrolysis system could be completely dissolved.

In some embodiments of the present disclosure, a reagent for adjusting the pH value to 1-2 is hydrochloric acid. In the present disclosure, there is no specific limitation on the concentration and addition amount of the hydrochloric acid, as long as the pH value could be adjusted to 1-2.

In some embodiments of the present disclosure, the method further comprises: subjecting a second hydrolyzed material liquid obtained after the second hydrolysis to a solid-liquid separation to obtain the first acidic wastewater and the crude product.

In some embodiments of the present disclosure, after obtaining the crude product, the method further comprises: subjecting the crude product to a purification treatment, and the purification treatment comprises the following steps: washing and drying the crude product to obtain 3,3′,4,4′-dicyclohexyltetracarboxylic acid.

In some embodiments of the present disclosure, a reagent for washing is water, and the water is deionized water. In the present disclosure, there is no specific limitation on the reagent for washing, as long as the product could be washed clean. In the present disclosure, there is no specific limitation on the parameters of drying, as long as the product could be dried to a constant weight.

In some embodiments of the present disclosure, washing the crude product further results in a second acidic wastewater.

The present disclosure also provides a method for treating an acidic wastewater, comprising:
mixing an acidic wastewater with an aluminum-iron mixed powder, and conducting a complexation reaction to obtain a complexation reaction system; and
mixing the complexation reaction system with a polyvinyl alcohol, and conducting a precipitation reaction to obtain a purified acidic water.

In the present disclosure, the acidic wastewater and an aluminum-iron mixed powder are mixed, and the resulting mixture is subjected to a complexation reaction to obtain a complexation reaction system.

In some embodiments of the present disclosure, the aluminum-iron mixed powder has a mass ratio of iron to aluminum of 1:(1-3), and preferably 1:2.

In the present disclosure, the acidic wastewater is the first acidic wastewater and/or the second acidic wastewater obtained by the preparation method described in the above technical solutions. In some embodiments of the present disclosure, a mass ratio of the acidic wastewater to the aluminum-iron mixed powders is in a range of 1:(0.001-0.02).

In some embodiments of the present disclosure, the complexation reaction is conducted at ambient temperature, i.e. neither additional heating nor additional cooling is required; the complexation reaction is conducted for 0.5-2 h.

In the present disclosure, the complexation reaction could remove organic matters in an acidic wastewater.

In the present disclosure, after obtaining the complexation reaction system, the complexation reaction system is mixed with a polyvinyl alcohol, and the resulting mixture is subjected to a precipitation reaction to obtain the purified acidic water.

In some embodiments of the present disclosure, the polyvinyl alcohol is polyvinyl alcohol 2499.

In some embodiments of the present disclosure, a mass ratio of the acidic wastewater to the polyvinyl alcohol is in a range of 1:(0.001-0.02).

In some embodiments of the present disclosure, the precipitation reaction is conducted at a pH value of 6-7, and a reagent for adjusting the pH value of the precipitation reaction is sodium hydroxide. In some embodiments of the present disclosure, the precipitation reaction is conducted for 0.5-2 h. In some embodiments of the present disclosure, the precipitation reaction is carried out under stirring.

In the present disclosure, the method further comprises: subjecting a precipitation reaction material liquid obtained after the precipitation reaction to a solid-liquid separation to obtain the purified acidic wastewater.

In some embodiments of the present disclosure, the solid-liquid separation is conducted by means of filtration.

In some embodiments of the present disclosure, the purified acidic wastewater has a chemical oxygen demand (COD) of 50-150 mg/L.

In the present disclosure, the precipitation reaction could remove organic matters in acidic wastewater.

In some embodiments of the present disclosure, after obtaining the purified acidic wastewater, the method further comprises: distilling the purified acidic wastewater to obtain a distillate and a solid of salts.

In the present disclosure, there is no specific limitation on the operation of the distillation, and any distillation parameters well known to those skilled in the art could be used.

In some embodiments of the present disclosure, the distillate has a COD of mg/L.

In some embodiments of the present disclosure, the solid of salts is sodium chloride; sodium chloride has a total organic carbon (TOC) of 10-50 mg/L.

The distillate obtained in the present disclosure reaches the class IV of Environmental Quality Standard for Surface Water, thereby greatly improving the energy saving and environmental protection of the preparation method.

The method for preparing 3,3′,4,4′-dicyclohexyltetracarboxylic acid and the method for treating an acidic wastewater provided by the present disclosure will be described in detail below in conjunction with examples, but they should not be understood as limiting the protection scope of the present disclosure.

### Example 1

200 g of high-purity 3,3′,4,4′-biphenyltetracarboxylic acid (with a purity of 99.8%), 20 g of industrial hydrochloric acid (with a mass percentage of 31.5%), and 1200 g of 1,3-propanediol were added into a four-neck bottle at ambient temperature, and heated to a temperature of 85° C. and held for 5 h until 3,3′,4,4′-biphenyltetracarboxylic acid was completely dissolved. The resulting material liquid was then cooled to ambient temperature and charged into a reaction kettle. A mixed catalyst (the mixed catalyst had a mass ratio of ruthenium-carbon catalyst to rhodium-carbon catalyst of 1:6, wherein the ruthenium-carbon catalyst had a mass percentage of ruthenium of 5%, and the rhodium-carbon catalyst had a mass percentage of rhodium of 3%) with a wet weight of 3.3 g and a dry weight of 1.48 g was added thereto, and then a feed port was closed and a nitrogen replacement was performed for 3 times. After the replacement was completed and the pressure was released to normal pressure, the obtained material liquid was heated to a temperature of 85° C., and subjected to a reaction. Continuous hydrogen flow was started to be introduced thereto, and the pressure in the kettle was controlled at 0.4 MPa until the hydrogen absorption was completed. The material liquid obtained after the hydrogen absorption was filtered, obtaining a recovered catalyst and a first filtrate. The recovered catalyst was recycled. 35 g of caustic soda flakes were added to the first filtrate and kept at 80-90° C. for 2 h. After the heat preservation was completed, the resulting material liquid was cooled and filtered, obtaining a second filtrate and a second solid. The second filtrate was recycled, and the second solid was dried and then dissolved in 450 g of water. After dissolution, the obtained solution of the second solid was adjusted to a pH of 1.5 by hydrochloric acid and filtered, obtaining a third filtrate and a third solid. The third solid was rinsed with 200 g of pure water, obtaining a wet product of 3,3′,4,4′-dicyclohexyltetracarboxylic acid and an acidic wastewater. The wet product of 3,3′,4,4′-dicyclohexyltetracarboxylic acid was dried, obtaining 204.2 g of a product, with a molar yield of 98.52% and a purity of 99.94%.

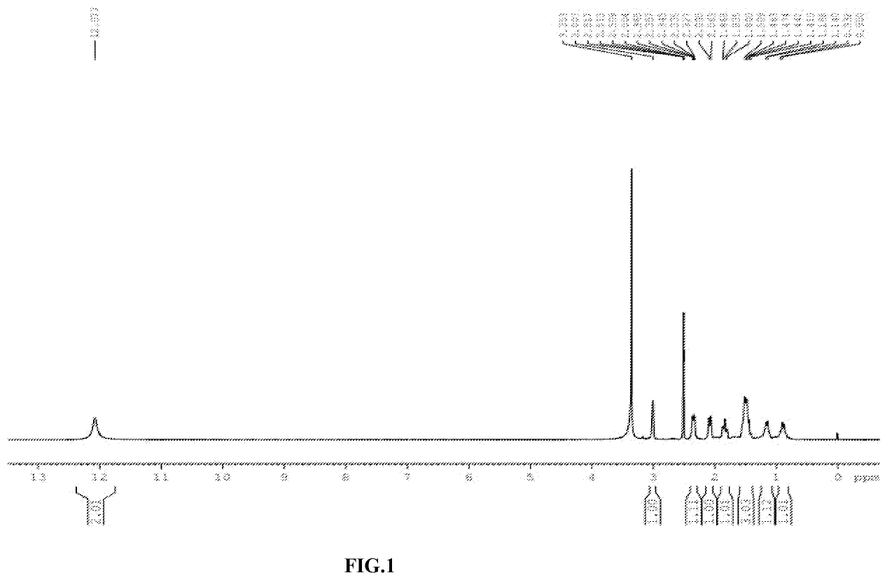

FIG. 1 is the NMR spectrum of the obtained 3,3′,4,4′-dicyclohexyltetracarboxylic acid. It can be seen from FIG. 1 that the product has a structure corresponding to 3,3′,4,4′-dicyclohexyltetracarboxylic acid, and is basically free of impurity.

2 g of iron powder and 3 g of aluminum powder were added to 634 g of acidic wastewater, and the resulting mixture was subjected to a complexation reaction for 0.5 h. Caustic soda flakes were added thereto to adjust the material liquid obtained after the complexation reaction to a pH of 7 and stirred for 30 min. 5 g of polyvinyl alcohol 2499 was added thereto, and stirred for 1 h. Then the resulting material liquid was filtered, obtaining a purified acidic wastewater with a COD of 98 mg/L and a residue.

The purified acidic wastewater was distilled until 85% of the solution was distilled out, and then cooled and filtered, obtaining 25.17 g of solid sodium chloride with a TOC of 45.7 mg/kg; a distillate distilled from the purified acidic wastewater has a COD of 26.4 mg/L, which reaches the class IV of Environmental Quality Standard for Surface Water.

### Example 2

200 g of high-purity 3,3′,4,4′-biphenyltetracarboxylic acid (with a purity of 99.8%), 25 g of industrial hydrochloric acid (with mass percentage of 31.5%), and 1200 g of 1,3-propanediol were added into a four-neck bottle at ambient temperature, and heated to a temperature of 90-100° C. and held for 3.5 h until 3,3′,4,4′-biphenyltetracarboxylic acid was completely dissolved. The resulting material liquid was then cooled to ambient temperature and charged into a reaction kettle. A mixed catalyst (the mixed catalyst had a mass ratio of ruthenium-carbon catalyst to rhodium-carbon catalyst of 1:6, wherein the ruthenium-carbon catalyst had a mass percentage of ruthenium of 5%, and the rhodium-carbon catalyst had a mass percentage of rhodium of 3%) with a wet weight of 3.1 g and a dry weight of 1.48 g was added thereto, and then a feed port was closed and a nitrogen replacement was performed for 3 times. After the replacement was completed and the pressure was released to normal pressure, the obtained material liquid was heated to a temperature of 85° C., and subjected to a reaction. Continuous hydrogen flow was started to be introduced thereto, and the pressure in the kettle was controlled at 0.4 MPa until the hydrogen absorption was completed. The material liquid obtained after the hydrogen absorption was filtered, obtaining a recovered catalyst and a first filtrate. The recovered catalyst was recycled. 40 g of caustic soda flakes were added to the first filtrate and kept at 85° C. for 2 h. After the heat preservation was completed, the resulting material liquid was cooled and filtered, obtaining a second filtrate and a second solid. The second filtrate was recycled, and the second solid was dried and then dissolved in 550 g of water. After dissolution, the obtained solution of the second solid was adjusted to a pH of 1-2 by hydrochloric acid and filtered, obtaining a third filtrate and a third solid. The third solid was rinsed with 100 g of pure water, obtaining a wet product of 3,3′,4,4′-dicyclohexyltetracarboxylic acid and an acidic wastewater. The wet product of 3,3′,4,4′-dicyclohexyltetracarboxylic acid was dried, obtaining 205.2 g of a product, with a molar yield of 99.0% and a purity of 99.96%.

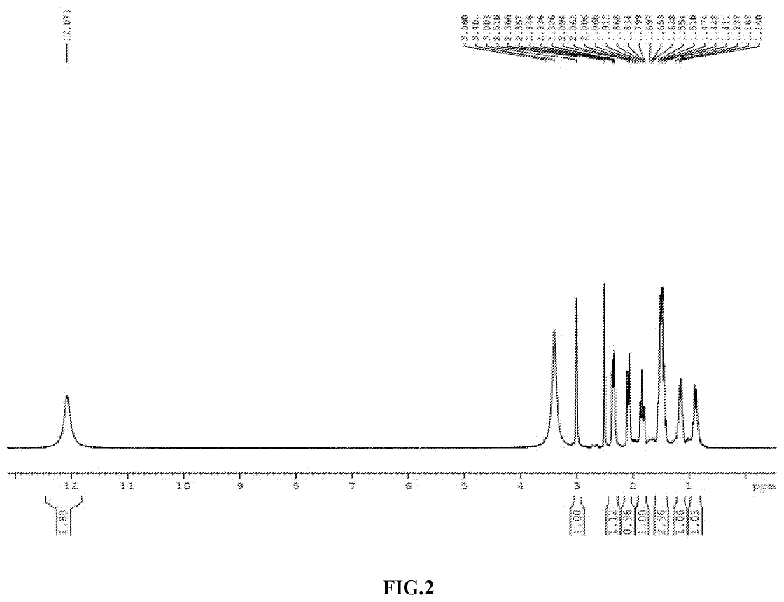

FIG. 2 is the NMR spectrum of the obtained 3,3′,4,4′-dicyclohexyltetracarboxylic acid. It can be seen from FIG. 2 that the product has a structure corresponding to 3,3′,4,4′-dicyclohexyltetracarboxylic acid, and is basically free of impurity.

4 g of iron powder and 6 g of aluminum powder were added to 628 g of acidic wastewater and the resulting mixture was subjected to a complexation reaction for 0.5 h. Caustic soda flakes were added thereto to adjust the material liquid obtained after the complexation reaction to a pH of 7.5 and stirred for 30 min. 7 g of polyvinyl alcohol 2499 was added thereto, and stirred for 1 h. Then the resulting material liquid was filtered, obtaining a purified acidic wastewater with a COD of 76 mg/L and a residue.

The purified acidic wastewater was distilled until 85% of the solution was distilled out, and then cooled and filtered, obtaining 23.1 g of sodium chloride solid with a TOC of 35.7 mg/kg, a distillate that distilled from the purified acidic wastewater has a COD of 21.4 mg/L, which reaches the class IV of Environmental Quality Standard for Surface Water.

### Example 3

200 g of high-purity 3,3′,4,4′-biphenyltetracarboxylic acid (with a purity of 98.5%), 25 g of industrial hydrochloric acid (with a mass percentage of 31.5%), and 1200 g of 1,3-propanediol were added into a four-neck bottle at ambient temperature, and heated to a temperature of 90-100° C. and held for 3.5 h until 3,3′,4,4′-biphenyltetracarboxylic acid was completely dissolved. The resulting material liquid was then cooled to ambient temperature and charged into a reaction kettle. A mixed catalyst (the mixed catalyst had a mass ratio of ruthenium-carbon catalyst to rhodium-carbon catalyst of 1:6, wherein the ruthenium-carbon catalyst had a mass percentage of ruthenium of 5%, and the rhodium-carbon catalyst had a mass percentage of rhodium of 3%) with a wet weight of 3.1 g and a dry weight of 1.48 g was added thereto, and then a feed port was closed and a nitrogen replacement was performed for 3 times. After the replacement was completed and the pressure was released to normal pressure, the obtained material liquid was heated to a temperature of 85° C., and subjected to a reaction. Continuous hydrogen flow was started to be introduced thereto, and the pressure in the kettle was controlled at 0.4 MPa until the hydrogen absorption was completed. The material liquid obtained after the hydrogen absorption was filtered, obtaining a recovered catalyst and a first filtrate. The recovered catalyst was recycled. 40 g of caustic soda flakes were added to the first filtrate and kept at 85° C. for 2 h. After the heat preservation was completed, the resulting material liquid was cooled and filtered, obtaining a second filtrate and a second solid. The second filtrate was recycled, and the second solid was dried and then dissolved in 550 g of water. After dissolution, the obtained solution of the second solid was adjusted to a pH of 1-2 by hydrochloric acid and filtered, obtaining a third filtrate and a third solid. The third solid was rinsed with 100 g of pure water, obtaining a wet product of 3,3′,4,4′-dicyclohexyltetracarboxylic acid and an acidic wastewater. The wet product of 3,3′,4,4′-dicyclohexyltetracarboxylic acid was dried, obtaining 198.2 g of a product, with a molar yield of 97.0% and a purity of 99.62%.

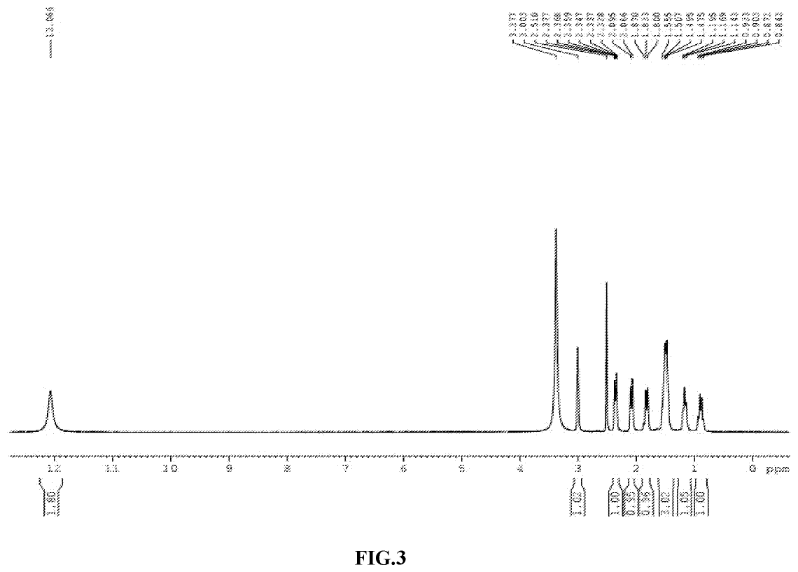

FIG. 3 is the NMR spectrum of the obtained 3,3′,4,4′-dicyclohexyltetracarboxylic acid. It can be seen from FIG. 3 that the product has a structure corresponding to 3,3′,4,4′-dicyclohexyltetracarboxylic acid, and is basically free of impurity.

4 g of iron powder and 6 g of aluminum powder were added to 634 g of acidic wastewater, and the resulting mixture was subjected to a complexation reaction for 0.5 h. Caustic soda flakes were added thereto to adjust the material liquid obtained after the complexation reaction to a pH of 7 and stirred for 30 min. 7 g of polyvinyl alcohol 2499 was added thereto, and stirred for 1 h. Then the resulting material liquid was filtered, obtaining a purified acidic wastewater with a COD of 186 mg/L and a residue.

The purified acidic wastewater was distilled until 85% of the solution was distilled out, and then cooled and filtered, obtaining 23.4 g of solid sodium chloride with a TOC of 42.8 mg/kg; a distillate distilled from the purified acidic wastewater has a COD of 22.4 mg/L, which reaches the class IV of Environmental Quality Standard for Surface Water.

### Comparative Example 1

200 g of high-purity 3,3′,4,4′-biphenyltetracarboxylic acid (with a purity of 98.5%), 25 g of industrial hydrochloric acid (with a mass percentage of 31.5%), and 1200 g of 1,3-propanediol were added into a four-neck bottle at ambient temperature, and heated to a temperature of 90-100° C. and held for 3.5 h until 3,3′,4,4′-biphenyltetracarboxylic acid was completely dissolved. The resulting material liquid was then cooled to ambient temperature and charged into a reaction kettle. A mixed catalyst (the mixed catalyst had a mass ratio of ruthenium-carbon catalyst to rhodium-carbon catalyst of 1:6, wherein the ruthenium-carbon catalyst had a mass percentage of ruthenium of 5%, and the rhodium-carbon catalyst had a mass percentage of rhodium of 3%) with a wet weight of 3.1 g and a dry weight of 1.48 g was added thereto, and then a feed port was closed and a nitrogen replacement was performed for 3 times. After the replacement was completed and the pressure was released to normal pressure, the obtained material liquid was heated to a temperature of 95° C., and subjected to a reaction. Continuous hydrogen flow was started to be introduced thereto, and the pressure in the kettle was controlled at 0.4 MPa until the hydrogen absorption was completed. The material liquid obtained after the hydrogen absorption was filtered, obtaining a recovered catalyst and a first filtrate. The recovered catalyst was recycled. 40 g of caustic soda flakes were added to the first filtrate and kept at 85° C. for 2 h. After the heat preservation was completed, the resulting material liquid was cooled and filtered, obtaining a second filtrate and a second solid. The second filtrate was recycled, and the second solid was dried and then dissolved in 550 g of water. After dissolution, the obtained solution of the second solid was adjusted to a pH of 1-2 by hydrochloric acid and filtered, obtaining a third filtrate and a third solid. The third solid was rinsed with 100 g of pure water, obtaining a wet product of 3,3′,4,4′-dicyclohexyltetracarboxylic acid and an acidic wastewater. The wet product of 3,3′,4,4′-dicyclohexyltetracarboxylic acid was dried, obtaining 182 g of a product, with a molar yield of 87.81% and a purity of 98.3%.

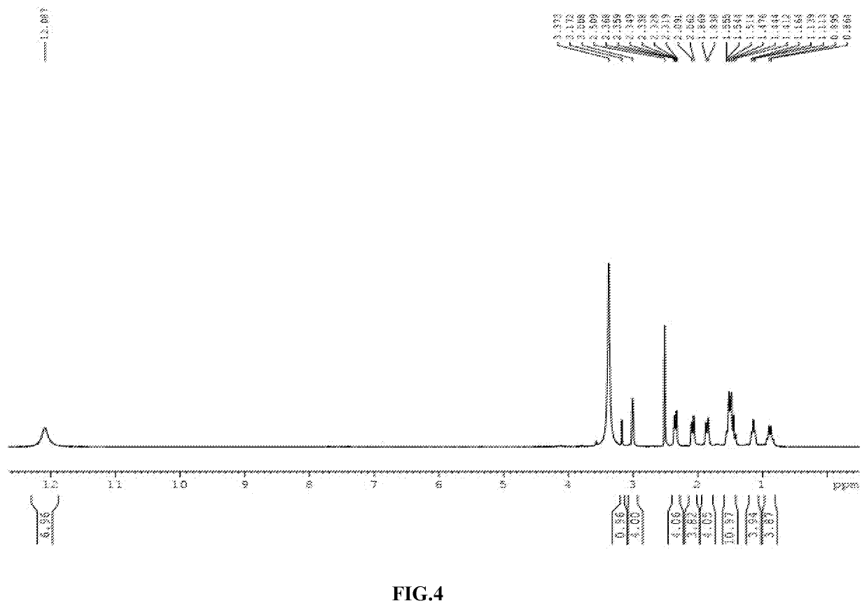

FIG. 4 is the NMR spectrum of the obtained 3,3′,4,4′-dicyclohexyltetracarboxylic acid. It can be seen from FIG. 4 that the product has a structure corresponding to 3,3′,4,4′-dicyclohexyltetracarboxylic acid.

4 g of iron powder and 6 g of aluminum powder were added to 628 g of acidic wastewater, and the resulting mixture was subjected to a complexation reaction for 0.5 h. Caustic soda flakes were added thereto to adjust the material liquid obtained after the complexation reaction to a pH of 7.5 and stirred for 30 min. 7 g of polyvinyl alcohol 2499 was added thereto, and stirred for 1 h. Then the resulting material liquid was filtered, obtaining a purified acidic wastewater with a COD of 352 mg/L and a residue.

The purified acidic wastewater was distilled until 85% of the solution was distilled out, and then cooled and filtered, obtaining 22.9 g of solid sodium chloride with a TOC of 67.9 mg/kg; a distillate distilled from the purified acidic wastewater has a COD of 143.2 mg/L.

The above are only preferred embodiments of the present disclosure. It should be pointed out that for those of ordinary skill in the art, without departing from the principle of the present disclosure, several improvements and modifications could be made, and these improvements and modifications should also be regarded as falling within the protection scope of the present disclosure.

What is claimed is:

1. A method for preparing 3,3′,4,4′-dicyclohexyltetracarboxylic acid, comprising the following steps:
mixing 3,3′,4,4′-biphenyltetracarboxylic acid and an alcohol, and subjecting the resulting mixture to an esterification reaction to obtain an esterification reaction system;
mixing the esterification reaction system and a ruthenium-rhodium mixed catalyst, and subjecting the resulting mixture to a hydrogenation reaction in a hydrogen atmosphere to obtain a hydrogenation reaction system; and
subjecting the hydrogenation reaction system to a hydrolysis reaction to obtain 3,3′,4,4′-dicyclohexyltetracarboxylic acid,
wherein the ruthenium-rhodium mixed catalyst is a mixed catalyst of a ruthenium-containing catalyst and a rhodium-containing catalyst, and the mixed catalyst has a mass ratio of a ruthenium-containing catalyst to a rhodium-containing catalyst of 1:6;
the ruthenium-containing catalyst is a ruthenium-carbon catalyst, and the ruthenium-carbon catalyst has a mass percentage of 5%;
the rhodium-containing catalyst is a rhodium-carbon catalyst, and the rhodium-carbon catalyst has a mass percentage of rhodium of 3%.

2. The method of claim 1, wherein 3,3′,4,4′-biphenyltetracarboxylic acid has a purity of not less than 99.8%.

3. The method of claim 1, wherein the alcohol comprises a monohydric alcohol and/or a dihydric alcohol, and the alcohol has a water content of not more than 2%.

4. The method of claim 3, wherein the monohydric alcohol comprises one or more monohydric alcohols selected from the group consisting of methanol, ethanol and isopropanol.

5. The method of claim 3, wherein the dihydric alcohol comprises 1,3-propanediol.

6. The method of claim 1, wherein the esterification reaction is conducted at a temperature of 80-100° C. and a pH value of 0.5-1.0 for 3-6 h.

7. The method of claim 1, wherein a mass ratio of 3,3′,4,4′-biphenyltetracarboxylic acid to the ruthenium-rhodium mixed catalyst is in a range of 1:(0.02-0.05).

8. The method of claim 1, wherein the hydrogenation reaction is conducted at a temperature of 80-90° C. and a pressure of 0.2-0.5 MPa.

9. The method of claim 1, wherein the hydrolysis reaction comprises:
subjecting a hydrogenation reaction material liquid obtained after the hydrogenation reaction to a solid-liquid separation to obtain a solid catalyst and a hydrogenation reaction system;
adjusting the hydrogenation reaction system to a pH value of 9-11, and subjecting the hydrogenation reaction system to a first hydrolysis to obtain a first hydrolysis system; and
adjusting the first hydrolysis system to a pH value of 1-2, and subjecting the first hydrolysis system to a second hydrolysis.

10. The method of claim 9, wherein the first hydrolysis is conducted at a temperature of 80-90° C. for 1-3 h.

11. The method of claim 9, wherein the method further comprises:
subjecting a second hydrolyzed material liquid obtained after the second hydrolysis to a solid-liquid separation to obtain a first acidic wastewater and a crude product; and
washing and drying the crude product to obtain a pure product of 3,3′,4,4′-dicyclohexyltetracarboxylic acid,
wherein a reagent for washing comprises water; and
the washing also results in a second acidic wastewater.

12. A method for treating an acidic wastewater, comprising the following steps:
mixing an acidic wastewater with a aluminum-iron mixed powder, and subjecting the resulting mixture to a complexation reaction to obtain a complexation reaction system; and
mixing the complexation reaction system with a polyvinyl alcohol, and subjecting the resulting mixture to a precipitation reaction to obtain a purified acidic water,
wherein the acidic wastewater comprises the first acidic wastewater and/or the second acidic wastewater obtained by the method of claim 11.

13. The method of claim 12, wherein the aluminum-iron mixed powder has a mass ratio of iron to aluminum of 1:(1-3).

14. The method of claim 12, wherein a mass ratio of the acidic wastewater to the aluminum-iron mixed powder is in a range of 1:(0.001-0.02).

15. The method of claim 12, wherein the complexation reaction is conducted for 0.5-2 h.

16. The method of claim 12, wherein a mass ratio of the acidic wastewater and the polyvinyl alcohol is in a range of 1:(0.001-0.02).

17. The method of claim 12, wherein the precipitation reaction is conducted at a pH value of 6-7 for 0.5-2 h.

18. The method of claim 12, wherein the method further comprises:
subjecting a precipitation reaction material liquid obtained after the precipitation reaction to a solid-liquid separation to obtain the purified acidic wastewater; and
distilling the purified acidic wastewater to obtain a distillate and a solid of salts.

19. The method of claim 7, wherein the hydrogenation reaction is conducted at a temperature of 80-90° C. and a pressure of 0.2-0.5 MPa.

20. The method of claim 16, wherein the precipitation reaction is conducted at a pH value of 6-7 for 0.5-2 h.
